(19) United States
(12) Patent Application Publication
(10) Pub. No.: US 2006/0063181 A1
(43) Pub. Date: Mar. 23, 2006

(54) Method for identification and quantification of short or small RNA molecules

(76) Inventors: Pamela J. Green, Newark, DE (US); Blake Mayers, Wilmington, DE (US); Cheng Lu, Newark, DE (US); Christian D. Haudenschild, San Francisco, CA (US); Shujun Luo, Castro Valley, CA (US)

Correspondence Address: Basil S. Krikelis;McCarter & English, LLP, Citizens Bank Center, 919 N. Market Street, Suite 1800, Wilmington, DE, 19801-3033, US

(21) Appl. No.: 11/204,903

(22) Filed: Aug. 15, 2005

Related U.S. Application Data
(60) Provisional application No. 60/601,747, filed on Aug. 13, 2004.
(60) Provisional application No. 60/602,221, filed on Aug. 17, 2004.

(51) Int. Cl. C12Q 1/68; C12P 19/34

(52) U.S. Cl. 435/6; 435/91.2

(57) ABSTRACT

A method of identifying and quantifying small RNA molecules comprising a) isolating RNA molecules; b) ligating RNA adapter molecules onto the isolated RNA molecules to form RNA template molecules; c) forming complementary DNA molecules by transcribing the RNA template molecules; d) amplifying the complementary DNA molecules; e) obtaining sequence information of the complementary DNA molecules (and thereby the RNA from which it was derived); and f) obtaining quantity information of the complementary DNA molecules, wherein the quantity information of the DNA molecules reflects the quantity of the isolated RNA molecules is provided. Included in the invention is the identification of RNA molecules between 15 and 30 nucleotides in length.

### CROSS-REFERENCE TO RELATED APPLICATIONS

This application claims the benefit of U.S. Provisional Application No. 60/601,747, filed Aug. 13, 2004; and U.S. Provisional Application No. 60/602,221, filed Aug. 17, 2004, the contents of which are incorporated by reference.

### RELATED FEDERALLY SPONSORED RESEARCH

The work described in this application was sponsored by National Science Foundation—Plant Genome #0110528 and #0439186 as well as the Department of Energy under contract #FG01-04ER04-01 and #DEFG02-04ER15541.

### SEQUENCE LISTING

This application explicitly includes the nucleotide sequences numbers: 1-5, which are also provided in the Sequence Listing contained on disc labeled with the following: Docket No. 99689-00011US; Applicant: Pamela J. Green, et al.,; Title: Method for Identification and Quantification of Short or Small RNA Molecules; Format: ASCII; SEQUENCE LISTING, Date Created: Aug. 15, 2005, Size: 2 kb; which is submitted herewith, and hereby incorporated by reference in its entirety.

### BACKGROUND OF THE INVENTION

One of the most exciting recent discoveries in biology is the complexity of transcribed sequences in eukaryotic genomes. Many RNA molecules do not encode proteins, but have independent functions as regulatory molecules. These transcripts that do not encode proteins but function directly as RNA molecules are called non-coding (ncRNAs). Non-coding RNAs are difficult to predict in the absence of experimental data, although recently developed comparative approaches may identify ncRNAs by differential patterns of conservation or mutation combined with predictions of secondary structure that may characterize ncRNAs.

Short and Small RNA Molecules

From published literature, it is known that small RNA molecules are produced by cleavage of longer molecules that are predicted to form ‘hairpin’ molecules or that have double-strand character. These small RNA molecules may cause transcriptional silencing by guiding a protein complex to sequences in the DNA or RNA being copied from it, that can base pair to the small RNA. This can render the DNA inactive. Small RNA can also guide protein complexes to other longer RNAs such as mRNAs, again by forming base-pairing interactions, and cause cleavage and accelerated degradation of the mRNAs. Alternatively, the small RNA molecules may reduce or prevent mRNA translation and thereby limit protein production. Any of these effects of small RNAs can produce a specific phenotype. The short length of the small RNAs, generally 15 to 30 nucleotides, is more than sufficient to specifically match nearly any given RNA encoded in a genome. In addition, this length is also short enough to make it possible for a single small RNA to match (and interact with) several members of a gene family that share short regions of similarity. These small RNA molecules do not need to match perfectly to their “target” molecules in order to direct the cleavage of the longer mRNA molecule. The small RNA molecules do not encode a protein, rather their effect results from a reduction in the mRNA abundance or protein abundance of the gene which is the “target”.

Published literature also demonstrates that there are two major types of small RNAs, known as small interfering RNAs (siRNAs) and microRNAs (miRNAs). Both sets of molecules are of a similar size, both are produced by cleavage of a longer double-stranded RNA molecule by a protein known as Dicer, an RNase III enzyme. These molecules have been identified in many sources. However, while the siRNAs and miRNAs are not easily distinguished by size, their biogenesis and sometimes their functional roles in biology are substantially different. The differences and similarities of siRNAs and miRNAs have been reviewed numerous times in the literature, as have been the mechanisms that endogenously produce these small RNA molecules.

Short RNA molecules refer here to those molecules that are less than 600 nucleotides and thus smaller than most mRNAs. They may be produced in an intact form or following processing from a larger molecule, with or without polyadenylation. Short RNA molecules may encode short peptides that have specific activities or they may be “noncoding” and exert their function as RNAs. Some short RNAs have known roles and structures such as 5S RNA, tRNA, snRNAs, and snoRNAs. Others are precursors of small RNAs or have been predicted by computational approaches or the experimental isolation of short RNAs. Most have yet to be identified because short RNAs are usually discarded during typical mRNA or small RNA isolation procedures.

Early methods for identifying these short or small RNA molecules focused on making longer “concatamers” of these molecules, and sequencing these concatamers using standard DNA sequencing methods. Using these methods, other research groups have identified more than 1900 distinct short or small sequences from the plant Arabidopsis thaliana.

Many of the known miRNAs function in flower development, and the current data suggests that the most common role for miRNAs is in development. It is also possible and probable that short and small RNAs play important roles in many other aspects of biology, such as abiotic and biotic stress. Because the discovery of these small RNAs has only occurred in the last 5 to 7 years, and because no methods prior to our invention permitted the large-scale characterization of these molecules, their ‘downstream’ role in many aspects of biology has been poorly explored, although the ‘upstream’ biochemical steps that produce these molecules are by now extremely well characterized.

Short or small RNAs have specific biological effects in many organisms. Prior to the invention of this method, it was slow, laborious and costly to identify and measure these RNA molecules.

There is a need for an efficient method to produce a set of many hundreds of thousands of individual sequences to, for example, produce a “library” of short or small RNAs. The abundance or frequency of occurrence of each distinct sequences from such a library is indicative of the quantity in the original tissue from which the RNA was obtained. By comparison of these sequences to genomic DNA sequence information, it would be possible to detect the full-length mRNA transcript that serves as a biochemical precursor to the small RNAs.

Quantitative measurements of small RNA sequences reveals valuable information concerning cell differentiation, gene expression, cell signaling responses and pathways, and disease state cell processes.

### SUMMARY OF THE INVENTION

In one aspect, the invention provides a method of identifying and quantifying short or small RNA molecules comprising a) isolating RNA molecules; b) ligating RNA adapter molecules onto the isolated RNA molecules to form RNA template molecules; c) forming complementary DNA molecules by transcribing the RNA template molecules; d) amplifying the complementary DNA molecules; e) obtaining sequence information of the complementary DNA molecules (and thereby the RNA from which it was derived); and f) obtaining quantity information of the complementary DNA molecules, wherein the quantity information of the DNA molecules reflects the quantity of the isolated RNA molecules is provided.

In other aspects of the invention, the step of isolating RNA molecules comprises isolating RNA molecules by acrylamide, or other suitable gel, isolation, or isolating RNA molecules by size, specifically isolating RNA molecules between 15 and 30 nucleotides in length or larger molecules of less than 600 nucleotides in length. Aspects of the invention include sequencing and quantifying RNA molecules less than 600 nucleotides, between 6 and 30 nucleotides, and between 21 and 24 nucleotides.

In another aspect of the invention, the step of ligating RNA adapter molecules onto the isolated RNA molecules comprises ligating a 5′ adapter sequence and a 3′ adapter sequence onto the isolated RNA molecules, the RNA adapter molecules comprising a restriction enzyme recognition site and a priming site for PCR amplification, specifically the RNA adapter molecules comprise a polynucleotide sequence of SEQ ID NO:1 (5′ adapter sequence) or SEQ ID NO:2 (3′ adapter sequence).

In an alternative aspect of the invention, the steps of obtaining sequence information and quantity information comprise performing a massively parallel signature sequencing (MPSS) method. More specifically, this aspect provides a method of designing a process for identifying and quantifying small RNA molecules comprising a) selecting RNA adapter molecules to ligate onto isolated small RNA molecules to form RNA template molecules, wherein the selected RNA adapter molecules form a portion of the RNA template molecules that flank a variable insert consisting of the tiny RNA, the RNA template molecules transcribing a cDNA insert comprising restriction enzyme sites, wherein the cDNA insert is cleaved to generate an overhang region on each end of the insert through digestion by the restriction enzyme; b) selecting a tag vector, wherein the vector has a cloning site that is complementary with the overhang region of the cDNA insert; c) amplifying the tagged inserts and loading them on microparticles containing the corresponding antitags; and d) sequencing the inserts by MPSS.

In an additional aspect of the invention, the adapter moieties also contain primer sites to allow PCR amplification to be carried out. In yet another aspect of the invention, a method of quantifying the relative expression of small RNA molecules is provided. The method comprises a) isolating small RNA molecules from a first sample; b) isolating small RNA molecules from a second sample; c) sequencing the isolated small RNA molecules by a known sequencing process; and d) comparing sequencing data of the small RNA molecules isolated from the first and the second samples and/or within the same sample.

In another aspect of the invention, a method of ascertaining small RNA sequences is provided comprises a) isolating small RNA molecules; b) sequencing the isolated small RNA molecules by a known sequencing process; and d) identifying small RNA sequences from the sequencing data of the isolated small RNA molecules.

Another aspect of the invention involves obtaining sequence and quantity information comprising the following steps: a) isolating small RNA molecules from a sample, b) ligating adapter sequences to the 5′ and 3′ ends of the RNA molecules, the adapter moieties comprising sites at the 5′ termini for reversible covalent attachment to a solid phase, primer sites for amplification, and restriction enzyme sites for initiation of sequencing to create a solid-phase cloning construct, c) covalently linking the construct to a solid-phase surface in the presence of covalently-linked primers corresponding to the primer sites in the adapters, d) amplifying the construct by the method of “bridge” amplification to generate solid-phase clonal colonies, and e) sequencing the small RNA portion of the colonies by MPSS or another parallel sequencing method.

### BRIEF DESCRIPTION OF THE DRAWINGS

FIG. 1 is a step by step overview of method for cloning of tiny or small RNAs. The endogenous RNA molecule is indicated in the figure, with each of the steps in the purification, cloning and preparation for sequencing indicated in the flowchart.

FIG. 2 is a scale showing bars that indicate the abundance of the small RNA, with the maximum height indicating >100 transcripts per million (TPM) and red bars indicating >500 TPM. The small RNAs are from an Arabidopsis flower library arrayed on the five Arabidopsis chromosomes. Chromosomes are indicated with numbers at left and a scale bar across the top shows the approximate length in megabasepairsVertical bars indicate the location of a small RNA and the position above or below the center line indicating the strand. Small RNAs duplicated in the genome are shown at all locations at which they match. The highest density of small RNAs on each chromosome corresponds to centromeric regions.

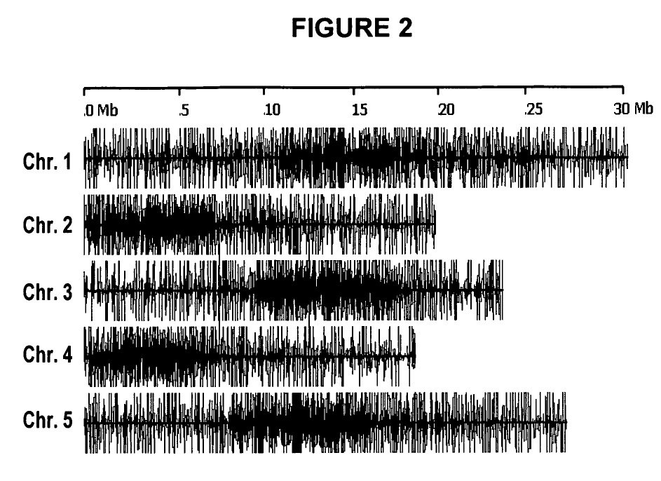

### DETAILED DESCRIPTION OF THE INVENTION

The present invention provides a method for isolating and cloning short and small RNA molecules. “Short RNAs” as used in this application are generally RNA molecules that are less than 600 nucleotides in size. Included within the class of short RNAs are “Small RNAs” which specifically refer to those RNAs of 6 to 30 nucleotides in size. Also presented herein is a method to efficiently sequence these RNA molecules, and quantify the abundance of particular RNA sequences. Importantly, this invention will contribute to the identification of new sources and targets of the short and small RNAs. Matching the large number of new short and small RNA molecules discovered by this invention to a genome is one way to accomplish this particularly when combined with the density of short and small RNAs in particular regions of the genome and with standard sequencing data from a sequencing system such as Massively Parallel Signature Sequencing (MPSS), data which may show inverse relationships. Data generated from this invention can be used to filter the output from existing computational tools used to identify source and target molecules or used to develop new tools that require larger numbers of sequences to be effective.

In its preferred from, the invention provides a way to identify and measure short or small RNAs from any organism by taking advantage of certain known methods in the art, combining a first stage of RNA isolation, with a second stage of MPSS. Such a combination was not trivial due to the need to optimize and customize each of the steps involved in the process in order to make the two stages work effectively together. Specifically, MPSS is not adapted to sequencing small RNA molecules. MPSS was originally designed to capture the fragment from the 3′-most DpnII site (or other restriction site) to the poly A tail of cDNA derived from mRNA transcripts. This required the presence of a defined restriction site, such as DpnII (GATC), or NlaIII (CATG) to allow capture and sequencing of the transcript end. MPSS was further modified to enable the capture uni-length signatures of up to 20 bases in length directly 3′ of the 3′-most DpnII (or other restriction) site, as well as the 20 bases directly adjacent to the polyA tail or the 5′-cap of mRNA transcripts.

Most short or small RNAs do not typically contain either a DpnII or NlaIII restriction site. Additionally, short or small RNAs are generally too short to enable the capture of 20-base signatures directly 3′ from their 5′ end, thus the existing MPSS method has been unavailable for sequencing short or small RNA molecules. In order to overcome this hurdle, unique RNA oligonucleotide adapters were designed to ligate onto the ends of short or small RNA molecules to permit processing by the MPSS method. The development of these unique adapter sequences, along with additional process developments, provide the method of this invention by which short and small RNA molecules can be sequenced and quantified by the MPSS method in addition to other sequencing methods known in the art.

The present invention provides a method of identifying and quantifying short and small RNA molecules. As mentioned earlier, short RNA molecules are typically defined as RNA molecules that are less than about 600 nucleotides in length, and more specifically, between about 25 to about 500 nucleotides in length. Small RNA molecules, on the other hand, while considered short RNAs, are specifically those RNA molecules between about 6 and about 30 nucleotides in length, and more specifically, between about 21 and about 24 nucleotides in length.

The method of identifying and quantifying small RNA molecules includes isolating RNA molecules from a sample source. An exemplary isolation process is detailed in the examples. Generally, short or small RNA molecules are isolated using standard techniques in the art. Any methods providing reliable size fractionation are suitable. Size fractionation on an agarose gel, or by PAGE fractionation are two acceptable methods of isolating the desired short RNA molecules for size. In isolating the RNA molecules, it is preferred that the RNA molecules be selected for size between 17 and 25 nucleotides in length, between 25 and 600 nucleotides in length, but any other range of desired length is acceptable. The short RNA molecules are then extracted and further isolated by standard techniques. The isolated RNA molecules are preferably single stranded with 90% purity by size.

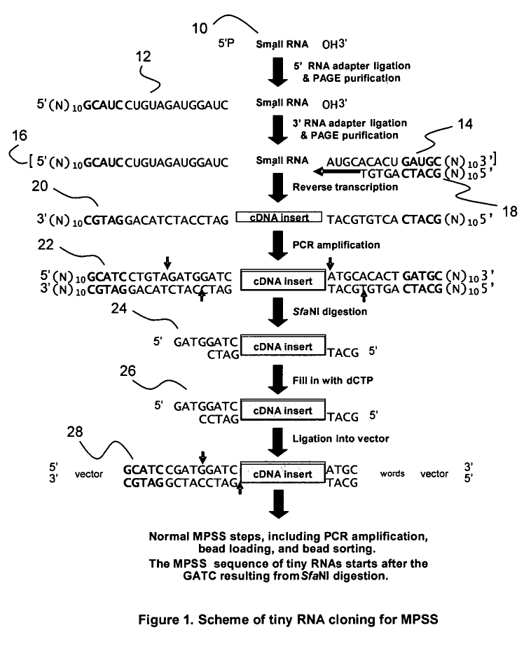

Once the desired population of short RNA molecules is isolated, RNA adapter molecules are ligated onto the ends of isolated RNA molecules to form RNA template molecules in which the small RNA insert is flanked by the adapters. The RNA adapter molecules are specifically designed adapters, as detailed below, that are covalently attached to the ends of the isolated single-stranded RNA molecule. While not necessary for success, the generally preferred process proceeds first by a 5′ ligation and then by a 3′ ligation. A schematic of this process is illustrated in FIG. 1. As shown in FIG. 1, the isolated small RNA molecules undergo ligation to a 5′ adaptor followed by ligation to a 3′ adapter. To improve the accuracy and signal-to-noise ratio of the sequence data, the RNA molecules are purified after each ligation step. These additional purification steps serve to eliminate unligated RNA sequences which may contaminate the sequencing results.

The 5′ and 3′ adapter molecules are each designed to provide a desired restriction enzyme cleavage site, priming sites for amplification, and sites for initiation of sequencing. The restriction enzyme cleavage sites are designed and/or selected for compatibility with the cloning and sequencing method of choice. It is generally preferred that the restriction sites be designed for Type II S restriction enzymes such as MmeI, BpmI, GsuI, and isochizomers thereof, among others. The sequencing initiation site can be a GATC sequence for initiation by DpnII cleavage, or by direct cleavage at a site generated by cleavage by an enzyme such as SfanI. Preferably, the adapters have RNA sequences that can be purchased from a commercial source, for example DHARMACON™, at the desired level of purity. As described later in the examples, SEQ ID NO:1 is an exemplary 5′ adapter sequence, and SEQ ID NO:2 is an exemplary 3′ adapter sequence for use with the SfaNI restriction enzyme and the MPSS methodology. While the sequence of the adapters for use in these methods are unique, the ligation of these adapters to the small RNA molecules can be accomplished through standard techniques.

Modification of adapter sequences (18) to avoid potential restriction sites or other deleterious sequences is an appropriate adjustment in the optimization of adapter sequence design. Lengthening the primer sequences (14) to cover more or all of the adapter is also an adjustment that may be employed to optimize primer sequences. Additionally, the PCR reactions (between 20 and 21) can be modified by incorporating methylated nucleotides, such as methyl C, to avoid inappropriate digestion by restriction enzymes used in the method.

FIG. 1 illustrates a preferred embodiment wherein a stepwise process of ligating an adapter 12 on to the 5′ end of an RNA molecule (labeled as “small RNA”) 10, followed by ligation of a companion adapter molecule 14 to the 3′ end. The 5′ and 3′ adapters ligated to the short or small RNA molecules forms a RNA template molecule 16. From this RNA template molecule, complementary DNA (cDNA) molecules 18 are formed by reverse transcribing the RNA template molecules. As shown in FIG. 1, the cDNA is preferably produced by reverse transcription. “Reverse transcription” means the transcription of RNA into complementary DNA. Reverse transcription generates a first strand of cDNA 20. As shown in FIG. 1, the “cDNA Insert” region of the cDNA molecule 20 is complementary to the original isolated RNA sequence 10. The cDNA 20 is amplified through an amplification process, such as the polymerase chain reaction (PCR) to generate double stranded product 22. Preferably, the amplification process of the cDNA does not alter the abundance of the population relative to the corresponding RNA molecules in the sample source. In order to prevent undesired amplification artifacts, the number of PCR amplification cycles should be minimized within the constraints of the methodology.

After amplifying the complementary DNA molecules, sequence information on the cDNA molecules can be obtained. While any sequencing method can be employed (as described later in this document), the most powerful and robust method currently available is MPSS. When using MPSS, the amplified product is digested with an appropriate restriction enzyme. As shown in FIG. 1, digestion by the restriction enzyme SfaNI forms a cDNA insert 24 that contains overhang regions that can be ligated into a tag vector selected for compatibility with the MPSS sequencing methodology.

Specifically, the restriction enzyme (SfaNI) recognizes its recognition site (the five nucleotide sequence ‘GTACT’ for SfaNI) and then cuts at its restriction site, indicated by arrows in FIG. 1 (for SfaNI, the cut leaves a four nucleotide 5′ overhang). While FIG. 1 illustrates the process using specific adapters designed for use with SfaNI as the restriction enzyme, the process may be performed using any adaptor sequence designed to complement a preferred restriction enzyme.

The adaptor sequences are designed to provide several functional features, including restriction enzyme recognition, primer docking site, sequencing initiation sites, as well as digestion ends that optimally provide high ligation efficiency to specially designed vectors for use in the sequencing process. The adaptor sequences and vector sequences are designed in tandem to provide compatible ends for cloning.

The ligation of the cDNA into the sequencing vector yields a product which can be further processed for traditional sequencing or a massively parallel sequencing method. In the figures and examples discussed below, the preferred method of sequencing is MPSS. The tagged inserts are amplified, digested to reveal the tags, loaded onto microparticles containing the corresponding antitags, and sequenced by MPSS, as described elsewhere.

Another method of massively parallel sequencing utilizes highly multiplexed clonal colonies of small RNA-containing constructs on a planar surface. In the colony approach purified small RNAs are ligated to adapters containing functionality for reversible immobilization on a solid surface, amplification via PCR or isothermal methods, and initiation of sequencing (via restriction cleavage) to yield template constructs for solid-phase cloning. The solid-phase cloning procedure is accomplished by covalently attaching the template construct via its 5′ terminus at a density suitable for generating colonies from single molecules. Primers corresponding to the amplification sequences are likewise covalently immobilized on the solid surface at a suitable density. Amplification, is carried out, for example, by PCR to produce double-stranded “bridge” intermediates which are subsequently denatured and repeatedly amplified by the same process until approximately 1000-2000 copies of each template is obtained per colony.

Sequence information may be derived through use of a web-based database of an MPSS library constructed from a genome library such as, for example, the Arabidopsis flowers. The location of potential mRNA MPSS signatures in such a genome can be plotted using data from available databases. For example, small RNAs may be densely clustered around a copia-like retrotransposon in Arabidopsis, and the small RNAs that are associated with the retrotransposon can be listed. Additionally, raw and processed abundance data for a specific library can be provided. The final calculated abundance level for each small RNA sequence in a tissue can be used to rank RNAs within the sample, or compare across samples. Small RNAs may target specific genes or intergenic regions within a complex region of the genome that contains numerous genes.

Sequencing of the colonies can be carried out by any number of methods, including sequencing by addition, pyrosequencing and MPSS. In the case of MPSS, template colonies are cleaved with a suitable restriction enzyme to create a specific site for hybridization of a sequencing initiation adapter. Subsequent sequencing steps are then carried out in a similar manner to the published MPSS methodology with the exception that imaging of the sequencing reactions is done on a solid surface instead on microparticles. More information regarding sequencing processes is provided later in this document.

Regardless of the method for collecting the sequence data, information on the quantity of the cDNA molecules, which reflects the quantity of the isolated RNA molecules is assessed if available from the data collected. The quantity information concerning the small RNA molecules reveals the abundance of a particular small RNA sequence within the tissue. Relative abundance information can be calculated among distinct small RNAs by counting the frequency of observations the sequence. This allows the small RNAs to be ranked by their relative abundance within the tissue, for example, to discover high or low abundance molecules. This discloses sequences that have a particular association with a characteristic of source. For example, sequences that have a high relative abundance in a disease-state sample compared with a non-diseased-state sample are associated with the disease response.

In another approach, the relative expression of small RNA molecules can be achieved by isolating small RNA molecules from a first sample, and isolating small RNA molecules from a second sample, followed by sequencing the isolated small RNA molecules by a massively parallel sequencing process, and comparing the sequencing data of the small RNA molecules isolated from the first and the second samples. This will identify molecules with differential frequencies in the two samples, and correlations of abundance may be made with treatments or conditions to identify small RNA molecules that may have a role in specific cellular responses.

Because the present method enables sequencing of short and small RNA molecules that are present in very small numbers in a population, it is possible to identify sequences that are not identifiable using more traditional methods. One example would be a comparison between the abundance of the miRNA* that is cleaved from the less abundant opposite strand of the larger hairpin miRNA precursor molecule shown in FIG. 1 of Reinhart et al., 2002 Genes and Devel. 16:1616-1626, incorporated herein by reference. Although the presence of tiny RNAs from both strands of the hairpins (i.e. miRNAs and miRNAs*) have been detected in rare cases, quantitative assessment has not been possible due to the previous lack of methods to sequence deeply enough into a population of tiny RNA molecules to measure tiny RNAs at such low abundance levels. Adapting the method for compatibility with the MPSS process enables sequencing of the low abundance small or tiny RNA molecules.

Sequencing

The methods of the invention are not limited to any particular sequencing method but can be used in conjunction with essentially any sequencing methodology which relies on successive incorporation of nucleotides into a polynucleotide chain. Suitable techniques include, for example, Pyrosequencing™, FISSEQ (fluorescent in situ sequencing), MPSS (massively parallel signature sequencing) and sequencing by litigation-based methods, some of which are described in more detail below.

As discussed above, one aspect of this invention is the use of massively parallel methods for the identification and quantification of short and small RNA sequences on a genome-wide basis. Preferably, the method allows the determination of the sequences of small RNA species in extremely low abundance in a cell by conducting a single experiment. This functionality identifies species that have importance in regulating various biological processes in the cell. Additionally, the method preferably exhibits a wide, dynamic range and high sensitivity enabling the quantitation of highly abundant as well as rare species. Accurate quantification of small RNA species, independent of abundance, provides insight to their role in regulating cellular processes. Also preferred is a method that provides an absolute measure of abundance, rather than relative quantitation as a ratio to a housekeeping or normalizing gene. Absolute abundance facilitates comparison of the small RNA abundances between samples and between experiments, and allows the data from different runs to be “banked” in a database and directly compared. Finally, in order to permit the discovery of new RNA species, particularly in organisms lacking complete genomic sequence coverage, the method preferably provides direct sequence readout, and is independent of prior sequence knowledge. Several methods for genome-wide sequence analysis have been described that demonstrate one or more of these performance features.

One alternative method of sequencing is set forth by Church et al. who have described a technology to generate highly multiplexed spherical polymerase colonies, or polonies, in which DNA template species are amplified in a polyacrylamide gel layer. This method uses the entrapment of DNA polymerase and immobilized acridyte-modified primers in a three-dimensional acrylamide matrix. By controlling the concentrations of primers in the amplification reaction, individual colonies containing to up to 108 copies of each template can be obtained. Church et al. indicate that on the order of tens of millions of colonies can be amplified on a single microscope slide, thus providing a suitable sampling depth for comprehensive genomic analysis. Polonies are sequenced in parallel via multiple cycles of primer extension with reversibly-labeled fluorescent oligonucleotides. To date, however, only short sequence reads of up to 8 base pairs have been obtained with polony mixtures of up to five different templates (Mitra, R., Shendure, J., Olejnik, J., Olejnik, E., and Church, G. Fluorescent in situ sequencing on polymerase colonies, Analytical Biochemistry 2003a; 320 (1):55-65). The technology has also been used for SNP genotyping (Mitra, R., Butty, V., Shendure, J., Williams, B., Housman D., and Church, G. Digital genotyping and haplotyping with polymerase colonies, Proc. Nat. Acad. Sci. USA 2003b; 100 (10): 5926-5931) and quantitation of RNA isoforms (Zhu, J., Shendure, J., Mitra, R., and Church, G. Single molecule profiling of alternative pre-mRNA splicing. Science 2003; 301: 836-838). Although potentially promising, this method has not yet been developed to the point of providing robust and quantitative performance and has not been extended to genome-wide analysis. (All references cited in this paragraph are incorporated herein by reference).

The sequencing methods of Mermod et al. (WO00/18957) and Adessi, C., et al. (Solid phase DNA amplification: characterization of primer attachment and amplification mechanisms, Nucleic Acids Res. 2000; 28 (20): e87.) are applicable as well. They have described a method of solid-phase PCR in which highly multiplexed DNA colonies derived from individual DNA fragments are created on the surface of a solid support. In this method, primer pairs and templates containing universal priming sites are immobilized on the surface of a functionalized glass slide at a density appropriate for the generation of discrete colonies. Amplification of the templates occurs by primer extension in a process called “bridge amplification” to create on the order of two thousand copies of each template per colony. This method is purported to yield colonies at a density of millions of features per mm², which is suitable for genome-wide analysis. Sequence analysis of the colonies can be carried out by traditional methods, such as sequencing by addition or MPSS. This promising method has not been reduced to practice for the sequence analysis of genomic fragments. (The references cited in this paragraph are incorporated herein by references).

Leamon et al., have described a method of highly multiplexed genomic DNA amplification in a low volume plate-based platform that is also applicable to this invention. PCR products derived from genomic fragments are attached to solid-phase beads, and sequencing of the fragments is carried out by synthesis using the Pyrosequencing™ technology. Such technology is applicable to the invention.

Other appropriate sequencing methods include multiplex polony sequencing (as described in Shendure et al., Accurate Multiplex Polony Sequencing of an Evolved Bacterial Genome, Sciencexpress, Aug. 4, 2005, pg 1 available at www.sciencexpress.org/4 Aug. 2005/Page1/10.1126/science.1117389, incorporated herein by reference), which employs immobilized microbeads, and sequencing in microfabricated picolitre reactors (as described in Margulies et al., Genome Sequencing in Microfabricated High-Density Picolitre Reactors, Nature, August 2005, available at www.nature.com/nature (published online 31 Jul. 2005, doi:10.1038/nature03959, incorporated herein by reference). In one aspect of the invention, these methods may be used to sequence the cDNA vectors to obtain sequence data on the isolated RNA sequences.

Massively Parallel Signature Sequencing (MPSS)

Massively Parallel Signature Sequencing (MPSS) technologies are powerful methods for the cloning, identification, and quantification of all expressed transcripts in a cell. The technologies enable comprehensive genome-wide digital transcriptional profiling, and have been established as the most powerful method for identifying poly adenylated transcripts. MPSS reveals the expression level of every gene expressed in a sample in a digital fashion by counting the number of individual molecules present. In a typical sample, a million or more transcripts are counted, providing quantitative expression data at single copy per cell levels. Accurate transcript measurement requires this depth of analysis because the typical cell contains more than 300,000 mRNA molecules and most, including many critical regulatory molecules are expressed at only a few copies per cell.

MPSS begins with the cloning of a fragment of up to 20 bases from every mRNA molecule in a given sample onto the surface of a 5 μm bead. Variations of the MPSS method have been described that enable the capture of fragments from different regions of mRNA transcripts. The original method captures the region from the terminal 3′ DpnII site to the polyA tail. The method has been modified to capture and identify internal unilength signatures of 17 or 20 bases from the 5′ end of the 3′most DpnII fragment. Finally, the method has also been adapted to capture up to 20 bases from either the 5′ end or 3′ end of full-length RNA transcripts. In each case, double-stranded cDNA is prepared from the RNA sample.

The process is best exemplified by the preparation of internal uni-length signatures. The cDNA is first digested with the restriction enzyme DpnII, which recognizes the sequence GATC. The 5′ end of the affinity purified 3′ end fragments, which extend from the DpnII site to the poly-A tail, are ligated to an adapter containing a type IIS restriction enzyme site. Subsequent cleavage with the type IIS restriction enzyme MmeI generates a constant-length signature of 20 base pairs in length. The 3′ end of these signatures are then ligated to a second adapter and directionally cloned into a tagging vector.

When cloned into the tagging vector, a unique DNA combitag sequence is attached to the signature fragment of cDNA derived from each mRNA. Combitags are 32-mer sequences consisting of minimally cross-hybridizing sets of eight four-mer nucleotide “words”. The tagged library is amplified, and the resulting cDNA is hybridized to beads, each of which is decorated with one hundred thousand identical antitags, which are oligonucleotide strands complementary to one of the combitags. Specific hybridization of the combitags with their corresponding antitags, results in each of the beads displaying amplified copies of one and only one starting mRNA molecule, with the DpnII end distal to the bead, and available for sequencing. The amplified cDNA copies on each bead originate from a single mRNA molecule. Thus, each bead is conceptually equivalent to a bacterial clone, with each clone (bead) harboring many copies of a single cDNA.

After hybridization, a minimum of one million beads are immobilized in a flow cell for sequencing biochemistry and imaging. The signature sequence on each bead is determined in parallel. The novel sequencing process involves repeatedly exposing four nucleotides by enzymatic digestion, ligating a family of encoded adapters, and decoding the sequence by sequential hybridization with fluorescent decoder probes.

Sequencing is initiated by ligation of an adapter molecule to the GATC single stranded overhang that has been re-exposed by enzymatic digestion. The adapter contains a recognition site for the type IIS restriction enzyme, BbvI. Subsequent enzymatic digestion with BbvI cuts the DNA at a position nine to 13 nucleotides away from the recognition site. This produces DNA strands with a four-base single stranded overhang immediately adjacent to the DpnII site. In order to determine which bases were revealed by the enzymatic cleavage, a set of 1024 encoded adapters are hybridized to the overhang. Encoded adapters contain all possible combinations of a four base single stranded overhang at one end, a single stranded decoding sequence at the other end, and an internal BbvI recognition site. One encoded adapter is ligated to its corresponding overhang on each bead. The identity of the ligated encoded adapter is then revealed by probing the decoding region sequentially with sixteen fluorescently-labeled decoder probes. Knowing the identity of the encoded adapter thus yields the identity of the four-base overhang in the signature. To collect additional sequence information, the cycle is repeated by cleavage with BbvI, which removes the first encoding adapter, and reveals the next four-base overhang for subsequent identification. Sequencing can also be carried out in multiple “frames” by the use of an indexing base positioned adjacent to the insert. In this way, MPSS results from more than one sample can be obtained in a single run.

The MPSS sequencing process is fully automated. Buffers and reagents are delivered to the beads in the flow cell via a proprietary instrumentation platform, and sequence-dependent fluorescent responses from the micro-beads are recorded by a CCD camera after each cycle. The 20-base-pair signature sequences, are constructed through this process from the images obtained at each cycle. Samples are routinely sequenced in two frames by the use of initiating adapters in which the restriction enzyme recognition site is offset by two bases. This ensures that signatures are not lost due to the presence of palindromes in one frame, although a small number of sequences with palindromes present in both sequencing frames will still be lost.

Comparison of the signature sequences with available databases identifies the region of the genome from which the signature was derived, or to which the small RNA sequence is targeted. Examples of small RNA signatures from a library made of flower tissue are shown after alignment with the Arabidopsis genome and presented in the Examples to follow. The Examples demonstrate the way in which the small RNA data reveal information about the genomic source and targets of these RNA molecules. Additionally, for genomes lacking the coverage of human or mouse, for example, MPSS provides direct sequence information for the discovery of novel genes and transcripts. The count of beads from each mRNA yields its frequency in the sample. The level of sensitivity provided by MPSS is critical for a variety of experiments because many important genes are expressed at low levels in the cell. MPSS has a routine sensitivity of a few molecules of mRNA per cell and the results are in a digital format that simplifies data management and analysis. MPSS results are particularly useful for generating the type of complete data sets that are useful in identifying functionally important genomic elements, such as tiny RNAs.

MPSS data have many uses. The expression levels of nearly all polyadenylated transcripts can be quantitatively determined; the abundance of signatures is representative of the expression level of the gene in the analyzed tissue. Quantitative methods for the analysis of tag frequencies and detection of differences among libraries have been published and incorporated into public databases for SAGE™ data and are applicable to MPSS data. The availability of complete genome sequences permits the direct comparison of signatures to genomic sequences and further extends the utility of MPSS data. The applicants have performed this comparison for Arabidopsis. Because the targets for MPSS analysis are not pre-selected (like on a microarray), MPSS data are able to characterize the full complexity of transcriptomes, and can be used for ‘gene discovery’. This is analogous to sequencing millions of ESTs at once, but the short length of the MPSS signatures makes the approach most useful in organisms for which genomic sequence data are available so that the source of the MPSS signature can be readily identified by computational means.

Additional information regarding MPSS technology can be obtained by reviewing the many publications on this subject, including U.S. Pat. Nos. 6,013,445, 5,846,719, and 5,714,330, all of which are incorporated herein by reference.

### EXAMPLES

### Example 1

### Low Molecular Weight (LMW) RNA isolation

Isolation of small or tiny RNA molecules was performed according to the following procedure:
1. Plant material from Arabidopsis thaliana (thale cress) was harvested and frozen in liquid nitrogen and ground to a fine powder.
2. Total RNA was isolated using TRIZOL (Invitrogen) reagent according to product protocol.
3. The total RNA (at least 500 ug) was dissolved in DEPC treated water.
4. mRNA and rRNA (high molecular weight RNAs) were precipitated in a solution of 10% PEG (MW=8000) (final concentration) and 0.5 M NaCl (final concentration).
5. The precipitating solution of RNA was mixed well and cooled in ice for 30 minutes.
6. The solution was centrifuged at max speed (˜11,000 g) for 10 minutes. The pellet contains the HMW RNAs and the supernatant contains the low molecular weight RNA molecules.
7. The supernatant was transferred to a microcentrifuge tube and 2.5 volumes of 100% EtOH was added to the supernatant. The tube was then cooled at −20° C. for at least 2 hours.
8. The microcentrifuge tube was centrifuged at max speed 11,000 g for 30 minutes at 4° C., forming a pellet containing LMW RNAs.
9. The resulting pellet was washed with 75% EtOH.
10. The pellet was dried and dissolved pellet in DEPC treated water.

### Example 2

### Purification of RNA 17-27mers from LMW RNA

1. Glass and spacers were prepared for pouring an polyacrylamide/urea gel.

2. A 15% polyacrylamide/urea gel was prepared. The components (see table below) were mixed and the solution was warmed to 37C in order to dissolve the urea. The solution was filtered through a nitrocellulose filter and cooled to room temperature.

Reagents

| | |
|---|---|
| Urea | 31.5 g |
| Acrylamide stock | 29.5 ml |
| 5 × TBE | 15 ml |
| Water | 8 ml |

3. 0.45 ml of a freshly prepared solution of 10% ammonium persulfate was added to the acrylamide solution and mixed well, using caution to avoid aeration of the solution.

4. 35 ul of TEMED was added to the above mixture, and the solution was mixed by gentle swirling. The solution was drawn into the barrel of a 50 ml syringe, and any air that entered the barrel was expelled. The nozzle of the syringe was introduced into the space between the two glass plates, and the space was filled almost to the top. The glass plates were place against a test-tube rack at an angle of 10 degrees, decreasing the chance of leakage and minimizing distortion of the gel. An appropriate comb was immediately added and the acrylamide was allowed to polymerize for 30 minutes at room temperature. The comb was removed and the wells were rinsed with 1×TBE. Prior to loading, the gel was run for 15-30 min at 400 V.

5. As much as LMW RNAs (in a volume of 10 ul) was loaded into each well as follows:
a. 2× loading dye which consists of an equal volume of formamide with dyes (0.05% xylene cyanol FF and 0.05% bromophenol blue) was added to the RNA solution and mixed well by vortexing, and then heated to 65° C. for 5 minutes.
b. The current was removed and the urea was washed from the well with 1×TBE.
c. Five to six slots were loaded with the heated LMW RNA.
d. 3 μg of 10 bp ladder was loaded in an unused lane as marker.

6. The gel was run until good separation of dyes.

7. The gel band corresponding to 17-27 nucleotides was sliced out of the gel and put into 15 ml tube and crushed.

8. Two volumes of RNA elution buffer (0.3 M NaCl) was added to the crushed gel slice (approximately 1.5 ml).

9. The elution buffer mixture was eluted overnight at room temperature with shaking.

10. The mixture was filtered through glass wool or Millex-HA 0.45 μm filter unit.

11. Chloroform extraction was preformed once.

12. Precipitation was preformed using 2.5 volumes of 100% EtOH with 2 μl glycogen (Ambion, 5 mg/ml). The mixture was cooled at −80° C. for 30 minutes.

13. The mixture was centrifuged at approximately 11,000 g max speed at 4° C. for 30 minutes, and the pellet washed with 75% EtOH, using as little EtOH as much as possible.

14. The washed pellet was allowed to air dry for about 5 minutes and then was resuspended in DEPC treated water (20 μl).

### Example 3

### 5′ Adaptor Ligation and Purification

1. Initiate a 5′ adaptor ligation reaction with the following components:
a. 5 μl 17-27 nt RNAs
b. 2 μl 200 μM 5′ RNA adaptor
c. 1 μl 10× Ligation Buffer
d. 2 μl T4 RNA ligase (Ambion, 5 u/μl)

2. Incubate at room temperature for 4-6 hours.

3. Stop reaction with 10 μl 2× Loading Dye.

4. Prepare a 10% denaturing polyacylamide gel. Prerun, then load into 2 lanes. Run gel until good separation of BB and XC.

5. Slice corresponding gel band (46-56 nt), put into 2 ml tube and crush.

6. Add two volumes of RNA elution buffer (0.3 M NaCl).

7. Elute overnight at RT with shaking.

8. Filter through glass wool or Millex-HA 0.45 μm filter unit (optional).

9. Extract with chloroform once.

10. Precipitate with 2.5 volumes of 100% EtOH with 2 μl glycogen (Ambion, 5 mg/ml). Cool at −80° C. for 30 minutes.

11. Spin at max speed (approximately 11,000 g) at 4° C. for 30 minutes, and wash with 75% EtOH to eliminate as much EtOH as possible.

12. Air dry approximately 5 minutes and resuspend in DEPC treated water (10 μl).

### Example 4

### 3′ Adaptor Ligation and Purification

1. Initiate a 3′ adaptor ligation reaction with the following components:
5 μl 5′ ligation product
2 μl 200 μM 3′ RNA adaptor
1 μl 10× Ligation Buffer
2 μl T4 RNA ligase (Ambion, 5 u/μl)
   Incubate at room temperature for 4-6 hours. Stop reaction with 10 μl 2× Loading Dye.

2. Prepare a 7.5% denaturing polyacylamide gel. Prerun, then load into 2 lanes. Run gel until good separation of BB and XC.

3. Slice corresponding gel band (70-80 nt), put into 2 ml tube and crush.

4. Add two volumes of RNA elution buffer (0.3 M NaCl).

5. Elute overnight at RT with shaking.

6. Filter through glass wool or Millex-HA 0.45 μm filter unit (optional).

7. Extract once with chloroform.

8. Precipitate with 2.5 volumes 100% EtOH with 2 μl glycogen (Ambion, 5 mg/ml). Cool at −80° C. for 30 minutes.

9. Spin at max speed (approximately 11,000 g) at 4° C. for 30 minutes. Wash with 75% EtOH. Eliminate as much EtOH as possible.

10. Air dry (approximately 5 minutes) and resuspend in DEPC treated water (10 μl).

### Example 5

### RT-PCR of Small RNAs Ligated with Adaptors

1. Using a siliconized tube, set up a reverse transcription reaction:
i. 5 μl ligated RNA
ii. 3 μl 100 μM RT-primer
iii. 5 μl DEPC treated water

2. Heat to 65° C. for 10 minutes, spin down to cool.

3. Add following in order:
i. 5 μl 5× first strand buffer (from invitrogen)
ii. 5.5 μl 2 mM of each dNTPs
iii. 3 μl 100 mM DTT
iv. 3 μl Superscript II RT (200 U/μl)
v. 1.5 μl RNase Inhibitor (from Ambion)

4. Heat to 48° C. for 3 min before adding RT.

5. Incubate at 44° C. for 1 hour.

6. Add 1 μl, 0.1M EDTA and 3.8 μl 1M KOH. Incubate at 90° C. for 10 minutes to degrade all the RNA.

7. Neutralize the reaction by adding 4 μl 1M HCl-Tris pH 1. Use the entire RT reaction for twleve 50 μl PCR amplification.

8. Set up 50 μl PCR reaction from the RT samples. Use new PCR tubes.
i. ×12
ii. 2.5 μl RT reaction 30
iii. 5 μl 10×PCR buffer 60
iv. 1.5 μl 50 mM MgCl 18
v. 1 μl 10 mM dNTPs 12
vi. 0.5 μl 100 μM 5′ PCR primer 6
vii. 0.5 μl 100 μM 3′ PCR primer 6
viii. 1 μl Taq (Invitrogen) 12
ix. 38 μl Water 456

9. 20-25 cycles of PCR (no hot start). 94C-1 min; 55C-1 min; 72C-1 min.

10. Analyze reaction with a 7.5% denaturing polyacrylamide gel. Take 5 μl from CR reaction, adding loading dye, heat well before loading. Run using the 10 bp ladder to follow bands. Use the SYBR Golds stain from Molecular Dynamics. You should see a good smear in the 75 nt size range.

11. Phenol/chloroform extraction once.

12. Chloroform extraction once.

13. Add NaCL to make 0.3 M, 2.5 volume 100% EtOH, with 2 μl glycogen (optional).

14. 75% EtOH washing, brief dry. Keep the pellet at −20° C.

### Example 6

### Exemplary Adaptor Sequences

1. Oligos for RNA Ligation

5′ RNA Adaptor:

SEQ ID NO. 1

GGU CUU AGU CGC AUC CUG UAG AUG GAU C:

3′ RNA Adaptor:

| | |
|---|---|
| AU GCA CAC UGA UGC UGA CAC CUG C: | SEQ ID NO. 2 |

RNA oligos were ordered from Dharmacon. Both adaptors were purified by PAGE.

2. Oligo for Reverse Transcription

RT-primer (DNA):

| | |
|---|---|
| GCA GGT GTC AGC ATC AGT GT: | SEQ ID NO. 3 |

3. Oligos for PCR Amplification

5′ PCR Primer (DNA):

| | |
|---|---|
| GGT CTT AGT CGC ATC CTG TA: | SEQ ID NO. 4 |

3′ PCR primer (DNA):

| | |
|---|---|
| GCA GGT GTC AGC ATC AGT GT: | SEQ ID NO. 5 |

### Example 7

### Massively Parallel Signature Sequencing

Using the MPSS sequencing system, the expression levels of the small or tiny RNA molecules can be quantitatively determined, because the abundance of signatures is representative of the expression level of the gene in the analyzed tissue. Comparisons of MPSS data across multiple tissues produce a quantitative description of the abundance or change in abundance for each RNA molecule. Because the expression level is determined by counting the abundance of a given MPSS signature, the technology is both sensitive to weakly expressed genes and unsaturated at high expression levels, giving the MPSS data a broad linear range and a high degree of accuracy. The power of this application of MPSS to measuring small or tiny RNA molecules is that prior quantification experiments depended on hybridization-based techniques such as Northern blots. With this method, it is possible to measure the amount of tiny RNAs so that their abundance can be compared with samples or among different samples.

Using MPSS sequencing, the first successful application of our invention produced 650,000 total sequences that comprised ˜58,000 distinct sequences. Of these distinct sequences, 50,000 were matched to the Arabidopsis genomic sequence. Of the 26 known Arabidopsis miRNAs, 22 were observed in our library.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A method of identifying and quantifying RNA molecules within a population of isolated RNA molecules, the method comprising:
a) ligating RNA adapter molecules onto the isolated RNA molecules to form RNA template molecules;
b) forming complementary DNA molecules by transcribing the RNA template molecules;
c) amplifying the complementary DNA molecules;
d) obtaining sequence information of the complementary DNA molecules; and
e) obtaining quantity information of the complementary DNA molecules, wherein the quantity information of the complementary DNA molecules reflects the quantity of the isolated RNA molecules.

2. The method of claim 1 wherein the isolated RNA molecules are isolated by gel electrophoresis.

3. The method of claim 1 wherein the isolated RNA molecules are isolated by size.

4. The method of claim 1 wherein the isolated RNA molecules are about 600 nucleotides or less in length.

5. The method of claim 1 wherein the isolated RNA molecules are between about 21 and about 24 nucleotides in length.

6. The method of claim 1 wherein the step of ligating RNA adapter molecules onto the isolated RNA molecules comprises ligating a 5′ adapter sequence and a 3′ adapter sequence onto the isolated RNA molecules.

7. The method of claim 6 wherein the method comprises purifying the RNA template molecules after ligating the 5′ adapter sequence onto the isolated RNA molecules.

8. The method of claim 6 wherein the method comprises purifying the RNA template molecules after ligating the 3′ adapter sequence onto the isolated RNA molecules.

9. The method of claim 1 wherein the RNA adapter molecules comprise a restriction enzyme recognition site and an amplification priming site.

10. The method of claim 9 wherein the RNA adapter molecules further comprise a restriction enzyme recognition site, a PCR primer recognition site, and a sequencing initiation site.

11. The method of claim 1 wherein the RNA adapter molecules further comprise an amplification priming site, functionality for covalent attachment at the terminus, and a sequencing initiation site.

12. The method of claim 1 wherein the RNA adapter molecules comprise a polynucleotide sequence of SEQ ID NO:1.

13. The method of claim 1 wherein the RNA adapter molecules comprise a polynucleotide sequence of SEQ ID NO:2.

14. The method of claim 1 further comprising a step of digesting the amplified complementary DNA molecules with a restriction enzyme.

15. The method of claim 14 wherein the restriction enzyme comprises SFaN1.

16. The method of claim 1 wherein the steps of obtaining sequence information and quantity information comprise performing a massively parallel signature sequencing (MPSS) method.

17. A method of identifying small RNA molecules within a population of isolated RNA molecules, the method comprising:
a) ligating RNA adapter molecules onto the isolated RNA molecules to form RNA template molecules;
b) forming complementary DNA molecules by transcribing the RNA template molecules;
c) amplifying the complementary DNA molecules; and
d) obtaining sequence information of the complementary DNA molecules.

18. A method of identifying and quantifying small RNA sequences, the method comprising:
a) isolating RNA molecules;
b) sequencing the isolated RNA molecules; and
c) identifying small RNA sequences from the sequencing data of the isolated RNA molecules
d) determining the quantity of each small RNA sequence.

19. The method of claim 18 wherein, prior to step b), further comprising the steps of:
a) ligating RNA adapter molecules onto the isolated RNA molecules to form RNA template molecules; and
b) forming complementary DNA molecules by transcribing the RNA template molecules.

20. The method of claim 19 further comprising the step of amplifying the complementary DNA molecules.


SEQUENCE LISTING (5 sequences)

SEQ ID NO: 1 - RNA, 28 nt, Artificial Sequence
  Transcription Template
ggucuuagucgcauccuguagauggauc

SEQ ID NO: 2 - RNA, 24 nt, Artificial Sequence
  Transcription Template
augcacacugaugcugacaccugc

SEQ ID NO: 3 - DNA, 20 bp, Artificial Sequence
  Primer Sequence
gcaggtgtcagcatcagtgt

SEQ ID NO: 4 - DNA, 20 bp, Artificial Sequence
  Primer Sequence
ggtcttagtcgcatcctgta

SEQ ID NO: 5 - DNA, 20 bp, Artificial Sequence
  Primer Sequence
gcaggtgtcagcatcagtgt